(19) United States
(12) Patent Application Publication
(10) Pub. No.: US 2021/0393735 A1
(43) Pub. Date: Dec. 23, 2021

(54) PHARMACEUTICAL COMPOSITION COMPRISING TEICOPLANIN

(71) Applicant: GALENUS G.H. AG, Hergiswil (CH)

(72) Inventors: Rita DOBMEYER, Hergiswil (CH); Kurt NABER, Straubing (DE)

(21) Appl. No.: 17/287,390

(22) PCT Filed: Oct. 20, 2019
(86) PCT No.: PCT/EP2019/078466

§ 371 Date: Apr. 21, 2021

(30) Foreign Application Priority Data
Oct. 23, 2018 (EP) 18202145.1

(51) Int. Cl. A61K 38/14

(52) U.S. Cl. CPC A61K 38/14; A61P 31/04

(57) ABSTRACT

The invention relates to a pharmaceutical composition or method for the treatment of microbial infections caused by bacteria, comprising administering to a patient separate therapeutically effective doses of teicoplanin or a pharmaceutically acceptable salt or prodrug thereof, wherein one or more loading doses of teicoplanin are administered, followed by administration of one or more maintenance doses of teicoplanin, wherein the amount of teicoplanin in each maintenance dose is half of the amount of each corresponding loading dose with about 10 to 14 hours intervals between each dose as well as pharmaceutical kit comprising separate pharmaceutical compositions with different therapeutically effective doses of teicoplanin.

### CROSS-REFERENCE TO RELATED APPLICATIONS

This application claims the benefit from the priority of European patent application EP 18202145.1 of Oct. 23, 2018; the entire of this application is hereby incorporated by reference.

### BACKGROUND OF THE INVENTION

### 1. Technical Field

The present invention relates to a pharmaceutical composition, wherein one or more loading doses of teicoplanin are administered followed by administration one or more maintenance doses of teicoplanin, whereby the amount of teicoplanin of each maintenance dose is half of the amount of the corresponding loading dose and all doses are administered with intervals of about 10 to 14 hours between each dose over the required length of treatment and to a ready-to-use kit of parts for the preparation of such a pharmaceutical composition.

### 2. Background Information

Before the introduction of antibiotics, patients suffering from acute microbial infections (e.g. tuberculosis or pneumonia) had a low chance of survival. For example, mortality from tuberculosis was around 50%. Although the introduction of antimicrobial agents in the 1940s and 1950s rapidly changed this picture, bacteria have responded by progressively gaining resistance to commonly used antibiotics. Now, every country in the world has antibiotic-resistant bacteria. Indeed, more than 70% of bacteria that give rise to hospital acquired infections in the USA resist at least one of the main antimicrobial agents that are typically used to fight infection (Coates et al, 2002 [1]).

The spectrum of antibacterial activity of teicoplanin is equivalent or superior to that of vancomycin. Gram-positive bacteria such as staphylococci (including methicillin-resistant strains), streptococci, enterococci and many anaerobic Gram-positive bacteria are susceptible to teicoplanin in vitro. The drug is equally efficacious against methicillin-resistant and -susceptible staphylococci (Campoli-Richards et al. 1990 [2]). Teicoplanin is also active against clinical isolates of Clostridium difficile from patients with hospital or community acquired Clostridium difficile infections (CDI) (Jamal, Rotimi 2016 [3])

Because of its very long terminal half-life of teicoplanin with a mean (±SD) of 157 (±93) hours (h) (Outman et al 1990 [4]) or with a median (range) of 168 (111-278) h (Antony et al 1991 [5]) the first 3-6 doses of 400 mg will be administered every 12 hours, followed by 400 mg every 24 h (Outman et al 1990 [4], Antony et al 1991 [5], Zhou et al 2018 [6]).

Accordingly, the conventional recommended dosage regimen is that after the early administration every 12 hours (bid) regimen (2-5 days) (loading dose) the same dose shall be administered only once daily thereafter (maintenance dose).

However, using this dosage regimen optimal trough levels (C(min)) of >20 mg/l will not be reached in the first days and are not kept during the maintenance dosing phase.

Therefore, the problem to be solved was to provide a dosage regimen of teicoplanin, which allows to reach optimal trough level (C(min)) of >20 mg/l without applying the theoretically ideal regimen of a continuous intravenous infusion, which, however, is not convenient in general practice and will not be adhered by the patience.

In the light of the above, it has been surprisingly found that a higher target trough level (C(min)>20 mg/l) can be achieved, if the maintenance dose of teicoplanin is administered with half of the amount as in the loading dose, but about every 12 hours instead of once daily.

### BRIEF SUMMARY OF THE INVENTION

Accordingly, in one embodiment of the present invention there is provided a pharmaceutical composition for the treatment of microbial infections caused by bacteria, comprising administering to the patient separate therapeutically effective doses of teicoplanin or a pharmaceutically acceptable salt or prodrug thereof, wherein one or more loading doses of teicoplanin are administered followed by administration one or more maintenance doses of teicoplanin, wherein the amount of teicoplanin of each maintenance dose is half of the amount of the corresponding loading dose and all doses are administered with intervals of about 10 to 14 hours between each dose over the required length of treatment.

In a further embodiment of the invention there is provided a pharmaceutical composition for increasing the tolerability of teicoplanin or a pharmaceutically acceptable salt or prodrug thereof, in a patient suffering from microbial infections caused by bacteria, comprising administering to the patient one or more loading doses of teicoplanin, which are followed by administration one or more maintenance doses of teicoplanin, wherein the amount of teicoplanin of each maintenance dose is half of the amount of the corresponding loading dose and all doses are administered with intervals of about 10 to 14 hours between each dose over the required length of treatment.

In a further embodiment, the invention provides a method for the treatment of microbial infections caused by bacteria in a patient comprising administering to the patient one or more loading doses of teicoplanin, which are followed by administration one or more maintenance doses of teicoplanin, wherein the amount of teicoplanin of each maintenance dose is half of the amount of the corresponding loading dose and all doses are administered with intervals of about 10 to 14 hours between each dose over the required length of treatment.

In a further embodiment, the invention provides a kit of parts for the preparation of a pharmaceutical composition according to this invention essentially consisting of:
(i) one or more separate doses of 400 mg to 800 mg of teicoplanin or a pharmaceutically acceptable salt or prodrug thereof;
(ii) one or more separate doses of 200 mg to 400 mg of teicoplanin or a pharmaceutically acceptable salt or prodrug thereof; and
(iii) instructions for treating a patient suffering from microbial infection caused by bacteria, said instructions comprising directions to administering said doses separately over the required length of treatment with intervals of 10 to 14 hours between every dose administration; or
(iv) one or more separate doses of 250 mg to 400 mg of teicoplanin or a pharmaceutically acceptable salt or prodrug thereof;
(v) one or more separate doses of 100 mg to 200 mg of teicoplanin or a pharmaceutically acceptable salt or prodrug thereof; and
(vi) instructions for treating a patient suffering from microbial infection caused by bacteria, said instructions comprising directions to administering said doses separately over the required length of treatment with intervals of 10 to 14 hours between every dose administration.

### DETAILED DESCRIPTION OF THE INVENTION

As used herein, the term “prodrug” relates to compounds, which are quickly transformed in vivo into pharmacologically active compounds. The design of prodrugs is generally studied in Hardma et al. (Eds.), Goodman and Gilman's The Pharmacological Basis of Therapeutics, 9th ed., pages 11-16 (1996).

An in-depth study is carried out in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987). In a preferred embodiment, colistimethate sodium (CMS) is a prodrug of colistin.

As used herein, the term “pharmaceutically acceptable salts” includes the metal salts or the addition salts which can be used in dosage forms. For example, the pharmaceutically acceptable salts of the compounds provided herein can be acid addition salts, base addition salts or metal salts, and can be synthesized from parent compounds containing a basic or acid residue by means of conventional chemical processes. Such salts are generally prepared, for example, by reacting the free acid or base forms of these compounds with a stoichiometric amount of the suitable base or acid in water or in an organic solvent or in a mixture of both. Non-aqueous media are generally preferred, such as ether, ethyl acetate, ethanol, isopropanol, or acetonitrile. Examples of acid addition salts include mineral acid additions salts such as, for example, hydrochloride, hydrobromide, hydroiodide, sulfate, nitrate, phosphate, organic acid addition salts such as, for example, acetate, maleate, fumarate, citrate, oxalate, succinate, tartrate, malate, mandelate, methanesulfonate and p-toluenesulfonate. Examples of alkali addition salts include inorganic salts such as, for example, ammonium salts and organic alkaline salts such as, for example, diethylamine, ethylenediamine, ethanolamine, N,N-dialkylenethanolamine, triethanolamine, glutamine and basic amino acid salts. Examples of metal salts include, for example, sodium, potassium, calcium, magnesium, aluminium and lithium salts.

As used herein, the term “pharmaceutically acceptable” relates to molecular entities and compositions that are physiologically tolerable and do not normally cause an allergic reaction or a similar adverse reaction, such as gastric discomfort, dizziness and the like, when administered to humans. As used herein, the term “pharmaceutically acceptable” preferably means that it is approved by a regulatory agency of the federal or state government or listed in the US pharmacopoeia or another pharmacopoeia, generally recognized for its use in animals, preferably in mammals and more particularly in human beings.

As used herein, the term “in combination with” or “co-administered” covers both separate and sequential administration of the antimicrobial agents. For example, when the agents are administered sequentially, either the teicoplanin or the polymyxin may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

As used herein, the term “teicoplanin” refers to an antibiotic used in the prophylaxis and treatment of serious infections caused by Gram-positive bacteria, including methicillin-resistant staphylococci, e.g. Staphylococcus aureus, and vancomycin-resistant enterococci, e.g. Enterococcus faecalis and Enterococcus faevium. It is a semisynthetic glycopeptide antibiotic with a spectrum of activity similar to vancomycin. Its mechanism of action is to inhibit bacterial cell wall synthesis.

Teicoplanin is as a rule a mixture of five compounds of the following formula

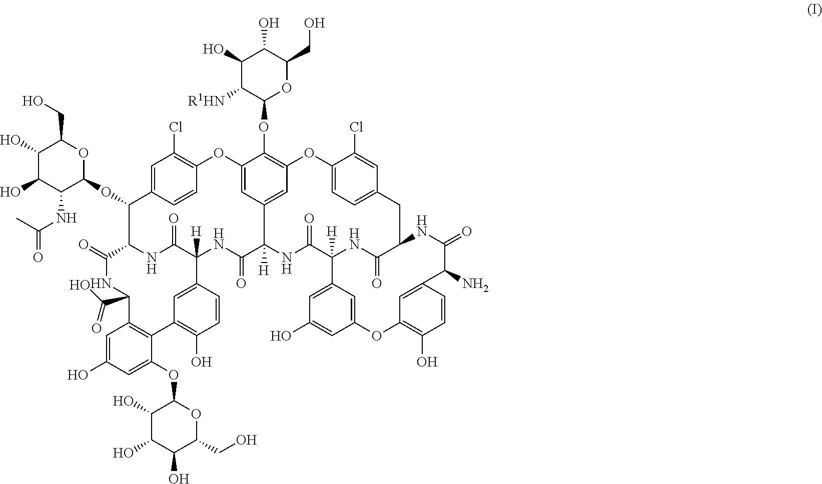

wherein
R¹ is a group selected from the formulae n-C₅H₁₁—CH═CH—(CH₂)₂—CO—, (CH₃)₂CH—(CH₂)₆—CO—, n-C₉H₁₉—CO—, C₂H₅—CH(CH₃)—(CH₂)₆—CO— and (CH₃)₂CH—(CH₂)₇—CO—

The pharmaceutical composition of the present invention is useful to treat microbial infections. In particular they may be used to kill also resistant microorganisms associated with microbial infections. References herein to the treatment of a microbial infection therefore include killing resistant microorganisms associated with such infections.

As used herein, “kill” means a loss of viability as assessed by a lack of metabolic activity. As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent. The metabolic activity of clinically latent microorganisms can be determined by several methods known to those skilled in the art; for example, by measuring mRNA levels in the microorganisms or by determining their rate of uridine uptake.

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection. Preferably, the term “microbial” in these contexts, means “bacterial.”

In a preferred embodiment of the present invention the microbial infection is caused by Gram-positive bacteria.

As used herein, the term “bacteria” (and derivatives thereof, such as “microbial infection”) includes, but is not limited to, references to organisms (or infections due to organisms) of the following classes and specific types:

Gram-positive cocci, such as Staphylococci (e.g. Staph, aureus, Staph, epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus), in particular methicillin-resistant Staphylococci; Streptococci (e.g. beta-haemolytic, pyogenic streptococci, such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic—Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus).

The combination of teicoplanin with polymyxin B or polymyxin E (colistin), preferably colistin, according to a preferred embodiment of the present invention is particularly beneficial in treating (multi)-drug-resistant ((M)DR) bacteria. It should be kept in mind that although a combination such as that claimed may initially be demonstrated to be functional in treating (M)DR strains, they can then be used in treating non-resistant strains.

The combinations of the present invention may be used to treat infections associated with any of the above-mentioned bacterial organisms, and in particular they may be used for killing multiplying and/or clinically latent microorganisms associated with such an infection. In one aspect the invention provides the use of teicoplanin in combination with polymyxin B or polymyxin E (colistin), preferably colistin, for treating microbial infections, particularly for killing clinically latent microorganisms associated with a microbial infection.

Particular conditions which may be treated using the pharmaceutical composition, the method or the combination of the present invention include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacilliary dysentry, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, eczema, empymea, endocarditis, endometritis, enteric fever, enteritis, epididymitis, epiglottitis, erysipelis, erysipelas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicameia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis; or infections with MSSA, MRSA, Staph. epidermidis, Strept. agalactiae, Strept. pyogenes, Escherichia coli,

Klebs. pneumoniae, Klebs. oxytoca, Pr. mirabilis, Pr. rettgeri, Pr. vulgaris, Ps. Aeruginosa, Haemophilis influenzae, Enterococcus faecalis and Enterococcus faecium.

It will be appreciated that references herein to “treatment” extend to prophylaxis as well as the treatment of established diseases or symptoms.

The following are preferred dosage regimens according to the invention:
(A) a regimen, with
   three to six loading doses of 5 to 7 mg/kg of teicoplanin,
   followed by at least five maintenance doses of 2.5 to 3.5 mg/kg of teicoplanin;
(B) a regimen, with
   one first loading dose of 10 to 12 mg/kg of teicoplanin,
   one to five additional loading doses of 5 to 7 mg/kg of teicoplanin,
   followed by at least five maintenance doses of 2.5 to 3.5 mg/kg of teicoplanin;
(C) a regimen, with
   five to ten loading doses of 10 to 12 mg/kg of teicoplanin,
   followed by at least five maintenance doses of 5 to 7 mg/kg of teicoplanin;

wherein in the regimens (A) to (C) all doses are administered with intervals of about 10 to 14 hours between each dose over the required length of treatment.

More preferred are the following dosage regimens according to the invention:
(D) a regimen, wherein
   the one or more, preferably one to six loading doses are in the range of 400 mg to 800 mg, and
   the one or more, preferably one to six following maintenance doses are in the range of 200 mg to 400 mg.
(E) a regimen, wherein
   the one or more, preferably one to six loading doses are in the range of 200 mg to 400 mg, and
   the one or more, preferably one to six following maintenance doses are in the range of 100 mg to 200 mg;

wherein in the regimens (D) and (E) all doses are administered with intervals of about 10 to 14 hours between each dose over the required length of treatment.

Preferably, the bacterial infections treated by the combination of teicoplanin and a polymyxin described herein are Gram-negative infections.

The active ingredients may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intradermal, intrathecal, intramuscular e.g. by depot and intravenous), rectal and topical (including dermal, buccal and sublingual) or in a form suitable for administration by inhalation or insufflation administration. The most suitable route of administration may depend upon the condition and disorder of the patient. Preferably, the compositions of the invention are formulated for parenteral, inhalative or topical administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

Formulations suitable for oral administration may be presented as discrete units such as capsules, cachets or tablets (e.g. chewable tablets in particular for pediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Topical compositions, which are useful for treating disorders of the skin or of membranes accessible by digitation (such as membrane of the mouth, vagina, cervix, anus and rectum), include creams, ointments, lotions, sprays, gels and sterile aqueous solutions or suspensions. As such, topical compositions include those in which the active ingredients are dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed may contain one or more components selected from the following list: a solubilising agent or solvent (e.g. a β-cyclodextrin, such as hydroxypropyl β-cyclodextrin, or an alcohol or polyol such as ethanol, propylene glycol or glycerol); a thickening agent (e.g. hydroxymethyl cellulose, hydroxypropyl cellulose, carboxymethyl cellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (e.g. a mixture of dihydrogen phosphate and hydrogen phosphate salts, or a mixture of citric acid and a hydrogen phosphate salt). Topical formulations may also be formulated as a transdermal patch.

The most suitable route of administration may depend upon the condition and disorder of the patient.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

Compositions for use according to the invention may be presented in a pack or dispenser device which may contain one or more unit dosage forms containing the active ingredients. The pack may, e.g. comprise a glass vial, a metal or plastic foil, such as a blister pack. Where the compositions are intended for administration as two separate compositions these may be presented in the form of a twin pack or a kit.

Compositions for inhalation will be administered by an inhaler (or puffer), which is a medical device used for delivering medication into the body via the lungs. Preferred inhalers are pressurized metered-dose inhalers (MDI), which are made up of 3 standard components—a metal canister, plastic actuator, and a metering valve, dry powder inhalers, which release a metered or device-measured dose of powdered medication and mechanically pressurized inhalers such as the Soft Mist Inhaler Respimat®.

Pharmaceutical compositions may also be prescribed to the patient in kit or “patient packs” containing the whole course of treatment in a single package, usually a blister pack or a pack of glass vials. Patient packs have an advantage over traditional prescriptions, where a pharmacist divides a patients' supply of a pharmaceutical from a bulk supply, in that the patient or the treating health professional always has access to the package insert contained in the patient pack, normally missing in traditional prescriptions. The inclusion of the package insert has been shown to improve patient compliance with the physician's instructions. The administration of the combination of the invention by means of a single patient pack, or patient packs of each composition, including a package insert directing the patient to the correct use of the invention is a desirable feature of this invention.

According to a further embodiment of the present invention there is provided a kit comprising at least one active ingredient of the combination according to the invention and an information insert containing directions on the use of the combination of the invention.

Dosages and formulations for the administration of colistin are described in the product label for Colomycin® which can be found at https://www.medicines.org.uk/cmc/mcdicine/1590

Dosages and formulations for the inhalative administration of colistin are described in the SPC for colistimethate sodium Colobreathe® which can be found at http://www.ema.europa.eu/docs/en_GB/document_library/EPAR_-_Product_Information/human/001225/WC500123690.pdf

Dosages and formulations for the administration of teicoplanin are described in the product label for Targocid® 400 mg powder for solution for injection/infusion or oral solution, which can be found at https://www.medicines.org/uk/emc/medicine/27321

The route of administration and dosage of polymyxin B and polymyxin E (colistin) is generally determined by the administering physician. Typically, polymyxin B and polymyxin E (colistin) is administered by topical, intramuscular, intravenous, intrathecal, inhalative or ophthalmic routes depending on the nature of the bacterial infection.

The administration of the combination of the invention by means of a single patient pack, or patient packs of each composition, including a package insert directing the patient to the correct use of the invention is a desirable feature of this invention. According to a further embodiment of the present invention there is provided a patient pack comprising at least one active ingredient of the combination according to the invention and an information insert containing directions on the use of the combination of the invention.

The amount of active ingredients required for use in treatment will vary with the nature of the condition being treated and the age and condition of the patient, and will ultimately be at the discretion of the attendant physician or veterinarian. In general however, doses of teicoplanin employed for adult human treatment will typically be in the range of 150 to 2,000 mg per day, preferably 200 to 1,500 mg per day.

The invention now being generally described, will be more readily understood by reference to the following Examples, which are included merely for purposes of illustration of certain aspects and embodiments of the present invention, and are not intended to limit the invention.

### EXAMPLES

### Example 1

Teicoplanin: Conventional Dosage Regimen as Compared to the Novel Dosage Regimen

Phase-1 Study in Healthy Subjects

Material and Methods

The study is performed as an open, cross over cohort-study after having received ethical approval and written consent by the participating 6 healthy subjects (3 males and 3 females). The principle study design of Outman et al (1990 [4]) is used with the following modification: 6 loading doses are followed by the maintenance dosing phase up to a total of 6 days.

In the first cross over comparative study, the following two dosage regimens are used:

Regimen A1: 6 loading dosages of 400 mg twice daily (bid) followed by 3 dosages of 400 mg once daily (total 6 days): total dosage 3600 mg

Regimen B1: first loading dose 800 mg followed by 4 dosages 400 mg bid, followed by 6 dosages 200 mg bid (total 6 days): total dosage 3600 mg.

In the second cross over comparative study, the following two dosage regimens are used:

Regimen A2: 6 loading dosages of 800 mg bid followed by 3 dosages of 800 mg once daily (total 6 days): total dosage 7200 mg

Regimen B2: 6 loading dosages of 800 mg bid followed by 6 dosages 400 mg bid (total 6 days): total dosage 7200 mg.

Results

In both, 1^{st} and 2^{nd} study Teicoplanin is generally well tolerated with both dosing regimens.

There is no evidence of phlebitis or renal, hepatic, or ototoxicity. Two subjects experience transient lightheadedness during the infusion of some, but not all, doses.

The overall basic pharmacokinetic parameters like t_{1/2} (157+93 h), total clearance (0.21+0.018 ml/min/kg), and renal clearance (0.19+0.015 ml/min/kg) do not differ significantly between the dosing regimens A and B in both studies.

In the 1^{st} study, there is, however, a significant (p<0.05) difference between the trough levels on the 1^{st} day between regimen A1 (8-15 mg/L) and B1 (18-25 mg/l) and between day 4 to 6 between regimen A1 (10-18 mg/L) and B1 (20-30 mg/L).

In the 2^{nd} study, there is also a significant (p<0.05) difference between the trough levels on the 1^{st} day between regimen A2 (14-24 mg/L) and B2 (25-34 mg/l) and between day 4 to 6 between regimen A2 (18-26 mg/L) and B2 (30-38 mg/L).

Conclusion

As compared with the conventional dosage regimen with the novel dosage regimen about 50% higher trough levels can be achieved at the 1^{st} day (shown in the first study only) and from day 4 on using the maintenance daily dosage regimen (400 mg in the 1^{st} study and 800 mg in the 2^{nd} study). Without increasing the total daily dosages the novel dosage regimen has advantage over the conventional dosage regimen to exhibit longer lasting effective plasma concentrations of teicoplanin, which might be crucial in case of borderline susceptible pathogens or in case of severe or difficult to reach infections, such as endocarditis or bone infections.

### REFERENCES

The publications cited hereinbefore and hereinbelow are hereby incorporated by reference in their entirety as if each individual publication is specifically and individually indicated to be incorporated by reference. In case of conflict, the present application, including any definitions herein, will control.
[1] Coates A, Hu Y, Bax R, Page C. The future challenges facing the development of new antimicrobial drugs. Nat Rev Drug Discov. 2002; 1(11):895-910.
[2] Campoli-Richards D M, Brogden R N, Faulds D. Teicoplanin. A review of its antibacterial activity, pharmacokinetic properties and therapeutic potential. Drugs. 1990 September; 40(3):449-86.
[3] Jamal W Y, Rotimi V O. Surveillance of Antibiotic Resistance among Hospital- and Community-Acquired Toxigenic Clostridium difficile Isolates over 5-Year Period in Kuwait. PLoS One. 2016 Aug. 18; 11(8):e0161411. doi: 10.1371/journal.pone.0161411.
[4] Outman W R, Nightingale C H, Sweeney K R, Quintiliani R. Teicoplanin pharmacokinetics in healthy volunteers after administration of intravenous loading and maintenance doses. Antimicrob Agents Chemother. 1990 November; 34(11):2114-7.
[5] Antony K K, Lewis E W, Kenny M T, Dulworth J K, Bradman M B, Kuzma R, Yuh L, Eller M G, Thompson G A. Pharmacokinetics and bioavailability of a new formulation of teicoplanin following intravenous and intramuscular administration to humans. J Pharm Sci. 1991 June; 80(6):605-7.
[6] Zhou L, Gao Y, Cao W, Liu J, Guan H, Zhang H, Shi Y, Lv W, Cheng L. Retrospective analysis of relationships among the dose regimen, trough concentration, efficacy, and safety of teicoplanin in Chinese patients with moderate-severe Gram-positive infections. Infection and Drug Resistance 2018:11 29-36.

1.-15. (canceled)

16. A method for the treatment of microbial infections caused by bacteria in a patient in need thereof, comprising:
administering to the patient in need thereof one or more loading doses of teicoplanin or a pharmaceutically acceptable salt or prodrug thereof;
subsequently administering to the patient one or more maintenance doses of teicoplanin or a pharmaceutically acceptable salt or prodrug thereof;
wherein the amount of teicoplanin or pharmaceutically acceptable salt or prodrug thereof of the maintenance dose is half of the amount of teicoplanin or pharmaceutically acceptable salt or prodrug thereof of the loading dose, and all doses are administered with intervals of about 10 to 14 hours between each dose over the length of treatment.

17. The method according to claim 16, wherein the loading dose comprises 400 mg to 800 mg of teicoplanin or a pharmaceutically acceptable salt or prodrug thereof, and the maintenance doses comprises 200 mg to 400 mg of teicoplanin or a pharmaceutically acceptable salt or prodrug thereof.

18. The method according to claim 16, wherein the loading dose comprises 200 mg to 400 mg of teicoplanin or a pharmaceutically acceptable salt or prodrug thereof, and the maintenance dose comprises 100 mg to 200 mg of teicoplanin or a pharmaceutically acceptable salt or prodrug thereof.

19. The method according to claim 16, wherein:
three to six loading doses of 5 to 7 mg/kg of teicoplanin or a pharmaceutically acceptable salt or prodrug thereof, and
at least five maintenance doses of 2.5 to 3.5 mg/kg of teicoplanin or a pharmaceutically acceptable salt or prodrug thereof,
are administered with intervals of about 10 to 14 hours between each dose over the length of treatment.

20. The method according to claim 16, further comprising:
administering to the patient in need thereof an additional dose of 10 to 12 mg/kg of teicoplanin or a pharmaceutically acceptable salt or prodrug thereof before administering the one or more loading doses of teicoplanin or a pharmaceutically acceptable salt or prodrug thereof;
wherein:
the additional dose of 10 to 12 mg/kg of teicoplanin or a pharmaceutically acceptable salt or prodrug thereof, and
one to five loading doses of 5 to 7 mg/kg of teicoplanin or a pharmaceutically acceptable salt or prodrug thereof,
followed by at least five maintenance doses of 2.5 to 3.5 mg/kg of teicoplanin or a pharmaceutically acceptable salt or prodrug thereof,
are administered with intervals of about 10 to 14 hours between each dose over the length of treatment.

21. The method according to claim 16, wherein:
five to ten loading doses of 10 to 12 mg/kg of teicoplanin or a pharmaceutically acceptable salt or prodrug thereof,
followed by at least five maintenance doses of 5 to 7 mg/kg of teicoplanin or a pharmaceutically acceptable salt or prodrug thereof,
are administered with intervals of about 10 to 14 hours between each dose over the required length of treatment.

22. The method according to claim 16, wherein the one or more loading doses of teicoplanin or a pharmaceutically acceptable salt or prodrug thereof are administered parenterally, and the one or more maintenance doses of teicoplanin or a pharmaceutically acceptable salt or prodrug thereof are administered parenterally or orally.

23. The method according to claim 16, wherein the microbial infection is caused by Gram-positive bacteria.

24. The method according to claim 16, wherein a resulting trough level (C₍ₘᵢₙ₎) of teicoplanin higher than 20 mg/L is obtained.

25. The method according to claim 16, wherein one or more loading doses and one or more maintenance doses are administered with intervals of about 12 hours between each dose over the length of treatment.

26. The method according to claim 16, wherein the microbial infection is caused by Gram-negative bacteria, and the method further comprises co-administering to the patient a pharmaceutical composition comprising an effective dose of a polymyxin.

27. A method of alleviating the symptoms of microbial infections caused by bacteria in a patient, comprising:
administering to the patient one or more loading doses of teicoplanin or a pharmaceutically acceptable salt or prodrug thereof; and
subsequently administering to the patient one or more maintenance doses of teicoplanin or a pharmaceutically acceptable salt or prodrug thereof;
wherein the amount of teicoplanin or pharmaceutically acceptable salt or prodrug thereof of the maintenance dose is half of the amount of teicoplanin or pharmaceutically acceptable salt or prodrug thereof of the loading dose, and all doses are administered with intervals of about 10 to 14 hours between each dose over the length of treatment.

28. A pharmaceutical kit comprising:
(i) one or more doses of 400 mg to 800 mg of teicoplanin or a pharmaceutically acceptable salt or prodrug thereof;
(ii) one or more doses of 200 mg to 400 mg of teicoplanin, or a pharmaceutically acceptable salt or prodrug thereof; and
(iii) instructions for treating a patient suffering from microbial infection caused by bacteria, the instructions comprising directions to administering the one or more doses separately over a required length of treatment with intervals of 10 to 14 hours between each dose administration.

29. A pharmaceutical kit comprising:
(i) one or more doses of 250 mg to 400 mg of teicoplanin, or a pharmaceutically acceptable salt or prodrug thereof;
(ii) one or more doses of 100 mg to 200 mg of teicoplanin, or a pharmaceutically acceptable salt or prodrug thereof; and
(iii) instructions for treating a patient suffering from microbial infection caused by bacteria, the instructions comprising directions to administering the doses separately over a required length of treatment with intervals of 10 to 14 hours in between each dose administration.
